(19) United States
(12) Patent Application Publication
(10) Pub. No.: US 2014/0296867 A1
(43) Pub. Date: Oct. 2, 2014

(54) ANATOMIC VAGINAL OCCLUDER

(71) Applicant: LSI SOLUTIONS, INC., Victor, NY (US)

(72) Inventors: Jude S. Sauer, Pittsford, NY (US); Evan R. Sutorius, Rochester, NY (US)

(73) Assignee: LSI Solutions, Inc., Victor, NY (US)

(21) Appl. No.: 13/854,528

(22) Filed: Apr. 1, 2013

(51) Int. Cl. A61B 17/42

(52) U.S. Cl. CPC A61B 17/42
USPC 606/119

(57) ABSTRACT

An anatomic vaginal occluder is constructed of soft, relatively compliant materials for atraumatic insertion through the vaginal opening. This device integrates at its distal end a horizontally oriented, elliptically shaped, occlusive prominence coupled with a larger vertically oriented, elliptical occlusive prominence, located more proximally on the device's longitudinal axis. The vaginal wall tissue is compressed radially and put under longitudinal tension completely circumferentially around the occlusive features to create a seal against gas loss.

### CROSS-REFERENCE TO RELATED APPLICATIONS

### None

### STATEMENT REGARDING FEDERALLY SPONSORED RESEARCH OR DEVELOPMENT

### None

### REFERENCE TO A “SEQUENCE LISTING”

Not applicable.

### BACKGROUND OF THE INVENTION

1. Field of the Invention

A potential space exists between the organs in the abdominal cavity and the inside walls of the abdomen. This potential space is usually collapsed and empty except for a trace of bodily fluids. Modern surgery strives to reduce the iatrogenic trauma from the lengthy skin incisions previously required for traditional open abdominal surgery. Over the past few decades, pelvic and abdominal surgeons have developed minimally invasive surgery techniques which access the abdominal (also called peritoneal) contents through small diameter ports, called cannulas. Cannula ports are passed through the skin and the abdominal wall musculature. Small imaging telescopes and narrow gauge surgical instruments can pass through these ports providing surgical access to the inside of the abdomen to view, diagnose and treat many of the common ailments that patients encounter. However, a collapsed peritoneal cavity (i.e., in its normal physiologic state) is too confined to permit safe visualization and tissue manipulation.

To open this potential space, thereby providing distance and working space between the abdominal wall and the internal structures, modern minimally invasive abdominal surgery routinely involves the infusion or installation of pressurized (and often moisturized) CO₂ gas. This use of pressurized CO₂ gas to expand the girth of the abdomen and expose internal structures is known as insufflation. The gas expanded abdominal cavity is called a pneumoperitoneum. Carbon dioxide, CO₂, is the most common gas routinely used throughout the world for this application as it is readily available and, most importantly, has proven quite safe. Since CO₂ is rapidly solubilized in human blood, any gaseous CO₂ that escapes the insufflated peritoneal cavity is promptly taken up by the blood in its non-gaseous form. Oxygen gas is not used for pneumoperitoneum. In addition to oxygen being explosive, oxygen has an extremely high risk of oxygen bubbles becoming trapped in the circulating blood. Aft is similarly problematic since it too remains in gaseous form too long in the blood. Aft is known to cause aft embolism and to impede cardiac pumping function by displacing blood in the chambers of the heart.

Routine laparoscopic insufflation pressure should not exceed approximately 15 mm of mercury (Hg). Higher pressures can reduce blood return to the heart by compressing intra-abdominal venous structures.

The abdominal space does not have any naturally occurring passageways that permit loss of gas or fluids directly out of the peritoneal cavity. Of course, the tubular structures of the abdomen such as the gastrointestinal tract, the urologic outflow tract and the vaginal canal do communicate with the outside world. However, the intact walls of these internal organs preclude any direct communication with the peritoneal cavity. To maintain approximately 15 mm Hg of pressure in the surgical field, large gas leaks out of the peritoneal cavity must be avoided. Modern cannulas incorporate seals to reduce gas loss. Laparoscopic instruments are designed to minimize the passage of pressurized gas through them.

Surgery often intentionally disrupts the continuity or integrity of the contained intraperitoneal space. For example, hysterectomy involves the amputation of the cervical portion of the uterus off of the forniceal (innermost) portion of the vaginal canal, leaving an open vaginal cuff. In open laparotomy for hysterectomy without pneumoperitoneum, this disruption of continuity is of little practical consequence, because exposure to the pelvic structures does not employ a pneumoperitoneum; the large incision used in open surgery enables visualization and direct manipulation by hand or retractors.

In laparoscopic surgery with an insufflated abdomen, an incision at the top of the vaginal canal (called a colpotomy) can open the pressurized peritoneal cavity to ambient pressure through the vaginal canal. If the vaginal passageway is not properly sealed at this point, positive pressure insufflation gas passes rapidly out causing the abdominal wall (ceiling) to collapse and halt the operation. Modern, commercially available transvaginal uterine manipulators (e.g., VCARE, CONMED Corporation, Utica, N.Y., U.S. Pat. No. 5,209,754, May 11, 1993, Ahluwalia; and The RUMI System Uterine Manipulator with the KOH Colpotomizer, CooperSurgical, Trumbull, Conn., U.S. Pat. No. 5,520,698, May 28, 1996, Koh) typically incorporate a feature to occlude gaseous passage out through the vaginal canal after colpotomy.

Extirpation or removal of the uterus from the patient is a key part of a total hysterectomy. Removal of the uterine specimen along with the uterine manipulation instrument and its vaginal occluder component, leaves the top (or cuff) of the vagina and the vaginal canal wide open for CO₂ insufflation gas loss. While the subsequent suturing of the vaginal cuff reestablishes a closed peritoneal cavity, cuff suturing requires a pneumoperitoneum. Therefore, to enable laparoscopic suturing, positive pressure insufflation must be reestablished after removal of the uterus and the uterine manipulator's vaginal occluder.

The human vaginal canal can be described as having five distinctive zones relevant for understanding the needs of a highly functional vaginal occlusive device. Starting from the outside of the patient, the first zone is the visible external female genitalia structures, including the labia, the clitoris and the external opening of the vaginal canal (introitus). Note the female urethra (the urinary outflow tract from the bladder) is located anterior to the vaginal opening. A drainage tube, called a Foley catheter, placed in the urethra is routinely used to remove urine from the bladder during pelvic surgery. The second zone is the vaginal canal passageway just inside the patient; this is the narrowest zone and is surrounded by the pelvic floor, a diaphragm of bony, muscular and fibrous tissue. Next is the third or middle zone of the vagina which is normally elliptical or oval in shape. With the patient in the supine position, (i.e., lying on her back), the middle elliptical shaped zone of the vagina typically has its longer axis oriented front to back or in the anterior to posterior plane (i.e., towards the umbilicus and sacrum). The fourth zone, the pre-forniceal zone, is also elliptical, but its long axis is generally oriented side to side or horizontal, essentially perpendicular to the third zone's elliptical axis. The fifth, innermost zone of the vaginal canal is known as the fornix or forniceal region. The fornix bulges radially slightly outward and then extends back directly toward the circumference or perimeter of the uterine cervix. A comprehensive scientific review article in 2006 states: “The average linear length of the vagina was 62.7 mm with a relatively large range (40.8-95 mm). It was noted that the width of the vagina varies throughout its length. The transverse diameter of the vagina is the highest at the level of the vaginal fornices (41.87 mm). The transverse diameter then progressively decreases from the cervical os (32.52 mm) to the pelvic flexure (27.97 mm), mid-lower vagina (27.21 mm), to the narrowest part of the vagina at the level of the vaginal introitus (26.15 mm).”

2. Description of Related Art

There are currently no known commercially available products specified to address this need for occluding the vaginal canal after removal of the uterus and uterine manipulator. Available uterine manipulators with their integrated vaginal occluders cannot be reinserted back into the patient after uterine specimen removal since these longer devices would pass through the full length of the remaining vagina and would impede access to the vaginal cuff.

Surgeons throughout the United States and elsewhere routinely use one of three of the following suboptimal approaches to temporarily block the opened vaginal canal for reestablishing insufflation prior to cuff suturing. Some surgeons intentionally leave part or all of the dissected uterine specimen in the vaginal canal to act as a plug. This incomplete specimen removal can be disorienting, bloody and distort the vaginal canal, making vaginal cuff suturing even more difficult. Other surgeons stuff sterile surgical gauze into a surgical glove for subsequent placement in vaginal canal. Besides being time consuming, awkward and unreliable, gloves and gauze can be readily captured by the suture needle and sewn into the vaginal cuff closure. A third common improvised vaginal occluder involves the use of a compliant, plastic bulb component removed from a sterile bulb syringe. These bulbs are often too large for smaller vaginal canals, leading them to fold into themselves, which causes gas leaks, or they are too small for larger canals so gas simply passes around the bulb.

All three of the above temporary vaginal occlusion approaches have routinely failed to reestablish and maintain insufflation. Since these makeshift devices also can pass completely inside of the patient's vaginal canal, patients have reportedly been discharged from the hospital with these currently used improvised vaginal occluders still retained in them. Foreign materials left in a postoperative patients can cause wound closure failure, extreme tissue damage and serious infection.

Several purported proprietary vaginal occlusive devices have been disclosed over the past few years. Pustilnik (Pub. No. US 2010/0168784 A1, Jul. 1, 2010) describes a bullet-shaped device with a circumferential inflatable balloon without any other customized safety features. This vaginal balloon appears not to be well differentiated from the balloon occluder feature from the decade-old RUMI KOH Uterine Manipulator technology. While the Pustilnik device's balloon may accommodate a variety of vaginal sizes, it offers no safety stop feature to keep the device in proper location for cuff suturing or preclude it from entering fully into the patient's vaginal canal. It also does not address the typical natural anatomic contours of the vaginal vault. A patent application by McDonald (Pub. No. US 2012/0203244 A1, Aug. 9, 2012), shows another vaginal occlusive device. A characteristic of this device is that it has a head, a shaft and a handle. While McDonald's device probably does not readily fall into the patient, it offers no feature to prevent it from falling out. No specialized safety, ergonomic or specific anatomic considerations are noted in the two disclosures above.

### BRIEF SUMMARY OF THE INVENTION

This invention provides an anatomic vaginal occluder as an improved means for hermetically occluding a patient's vaginal canal during surgery. This device is designed to snugly fit and fill the natural contours of the vaginal canal to block this passageway against insufflation gas loss after a colpotomy. Throughout this disclosure, all descriptions regarding anatomic orientation reference the patient lying on her back in the surgically supine position.

This anatomic vaginal occluder is constructed of soft, relatively compliant materials for atraumatic insertion through the vaginal opening. This device integrates at its distal end a horizontally oriented, elliptically shaped, occlusive prominence coupled with a larger vertically oriented, elliptical occlusive prominence, located more proximally on the device's longitudinal axis. The vaginal wall tissue is compressed radially and put under longitudinal tension completely circumferentially around the occlusive features to create a seal against gas loss. These prominences acting in unison exert outward radial compressive forces onto the inner lining of the vaginal canal in a shape consistent with the vaginal canal itself. With these occlusive prominences aligned perpendicularly to each other, the tubular vaginal tissue is pushed in opposite directions to increase stretching and to draw the tissue tightly against the occlusive prominences. The distal horizontal prominence helps orient the vaginal tissue into the preferred horizontal shape to optimize the suture wound closure in the correct anatomic plane.

The unique shape of this surgical device enables its easy insertion into most of the vaginal canals encountered in modern surgery. Its size and shape enables this device to fit into very small diameter vaginal passageways, like those found in a post-menopausal woman who has never experienced a vaginal delivery, and also very large vaginal canals, as found in some women who have experienced multiple vaginal deliveries.

No additional devices are required for anatomic vaginal occluder insertion. Only surgical lubrication is used. The operator's fingers act as an obturator and structural support mechanism during device insertion and orientation. During device installation, the operator's fingers are simply inserted into the opening in the occluder's proximal end and passed through its inner hollow space stopping at its closed distal end.

The anatomic vaginal occluder integrates a stop feature that is sized to reduce the risk of too deep or complete device insertion into the vaginal canal. By engaging the structures around the vaginal opening, this stop feature precludes the device from entering too deeply into the vaginal canal and interfering with vaginal cuff suturing. Without an effective stop feature, inadvertent placement of an indwelling vaginal occluder fully within the patient can lead to the device being left in the patient after the procedure. The device's asymmetric profile also prevents it from simply rolling off of the surgical table prior to use, as occurs frequently with symmetric cylindrical surgical devices.

The anatomic vaginal occluder's design makes it easy to install with essentially no “learning curve” required. It uses the known anatomic features of the pelvic floor to ensure the device stays positioned as intended in the vaginal vault. The pelvic floor surrounding the inside of the vaginal opening circumferentially engages the device between the stop ridge and the proximal occlusive prominence to prevent over insertion and the device falling out, respectively. The device is intended to reduce the risk of having its distal end inadvertently sutured into the vaginal cuff closure. The integrated stop ridge precludes the distal end from penetrating too near the vaginal cuff. In addition, the contour of the generally rounded and smooth closed distal end and the compliant yet firm plastic material reduces the opportunity for surgical needle puncture. The grip surfaces on the proximal device, which protrude outside of the patient, facilitate easy grasping of the device for removal. The proximal end of the device remains readily apparent outside of the external vaginal opening to ensure its removal at the appropriate time. This device is designed to provide adequate clearance for a Foley urinary drainage catheter commonly used in pelvic surgery.

A sterile inexpensive device that does not require re-cleaning is the most convenient option for the end user. Simple, relatively inexpensive manufacturing techniques may be employed for construction of this preferably single-patient-use device. Acceptable surgical device manufacturing techniques for making this technology could include blow molding, thermoplastic injection molding or dip molding.

No balloon filling or other instruments are required with the present invention. This novel option obviates the awkwardness of leaving the amputated uterine specimen in the vaginal canal as a plug, or the risks of placing a gauze-packed glove near the suturing site or the often ineffective use of a sterile bulb from a bulb syringe.

### BRIEF DESCRIPTION OF THE SEVERAL VIEWS OF THE DRAWING(S)

The novel aspects of the invention are set forth with particularity in the appended claims. The invention itself together with further objects and advantages thereof may be most readily understood by reference to the following detailed description of the invention taken in connection with the accompanying drawings in which:

FIG. 1 is a distally oriented perspective view of the anatomically shaped vaginal occluder showing the closed distal end that first enters the vaginal canal;

FIG. 2 is a side view of the anatomic vaginal occluder presented in FIG. 1;

FIG. 3 is a front view of the distal end of the anatomic vaginal occluder presented in FIG. 1;

FIG. 4 is a top view of the anatomic vaginal occluder presented in PG. 1 taken along lines 4-4 of FIG. 2;

FIG. 5 is a rear view of the anatomic vaginal occluder presented in HG. 1 taken along lines 5-5 of FIG. 2;

FIG. 6 is a proximally oriented perspective view of the anatomically shaped vaginal occluder presented in FIG. 1 showing the open proximal end that accepts the operator's fingers;

FIG. 7 is a cross sectional view of the anatomic vaginal occluder taken along lines 7-7 of FIG. 1 with the operator's fingers inserted for use;

FIG. 8 is a side view of the female pelvis sectioned along the midline (also known as a sagittal view) showing a cross-section of the vaginal canal and uterus;

FIG. 9 is a side view of the female pelvis sectioned along the midline showing the anatomic vaginal occluder inserted into vaginal canal with the uterus removed;

FIG. 10 is a schematic front view illustrating a human vagina and uterus;

FIG. 11 is a schematic front view illustrating a vaginal canal with an inserted anatomic vaginal occluder. The uterus is shown ghosted to indicate that it has been removed.

### DETAILED DESCRIPTION OF THE INVENTION

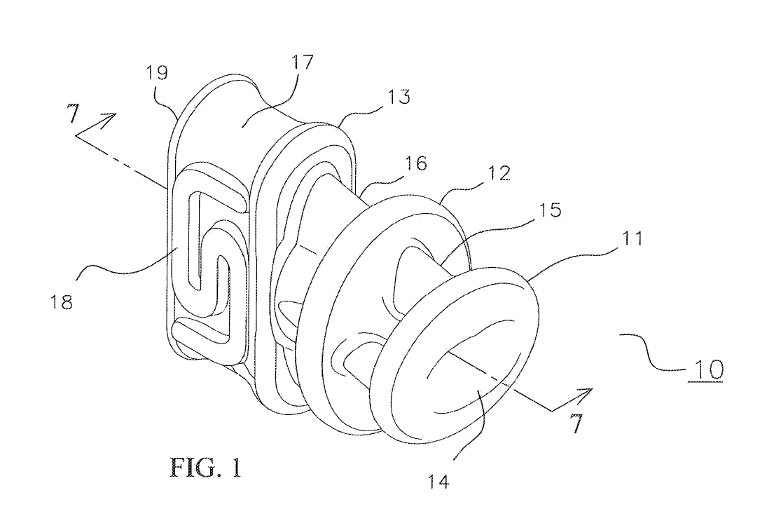

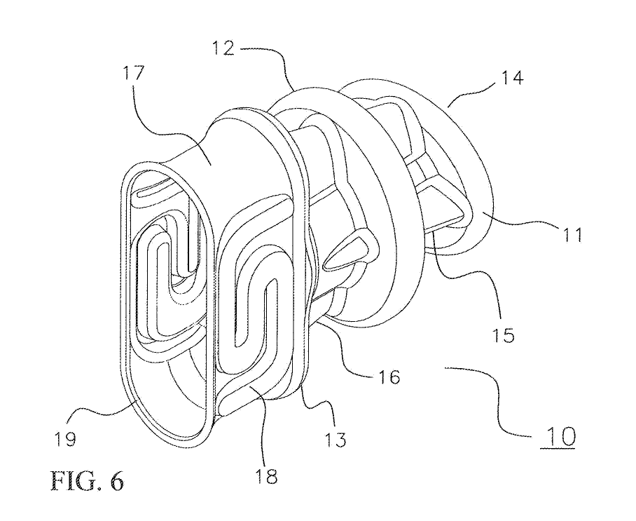

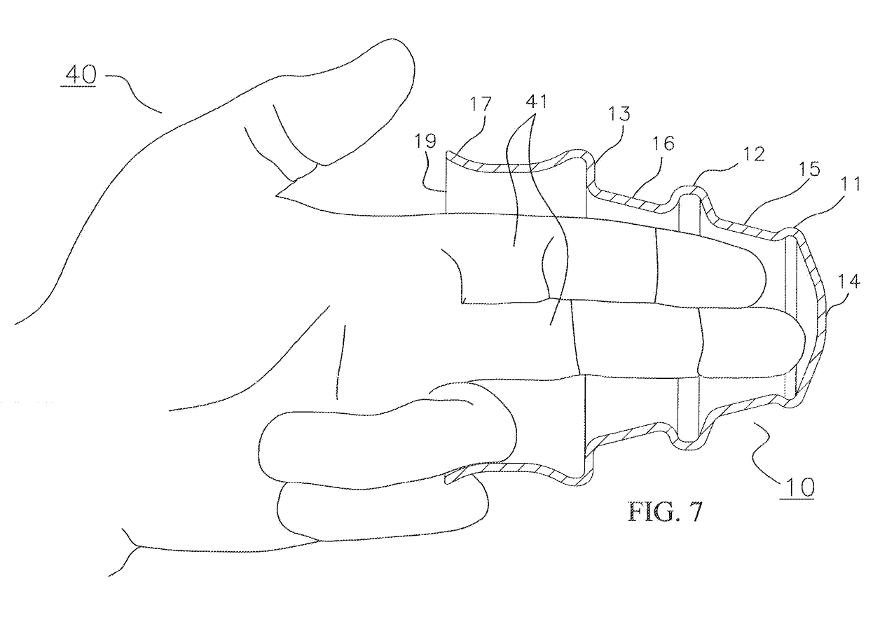

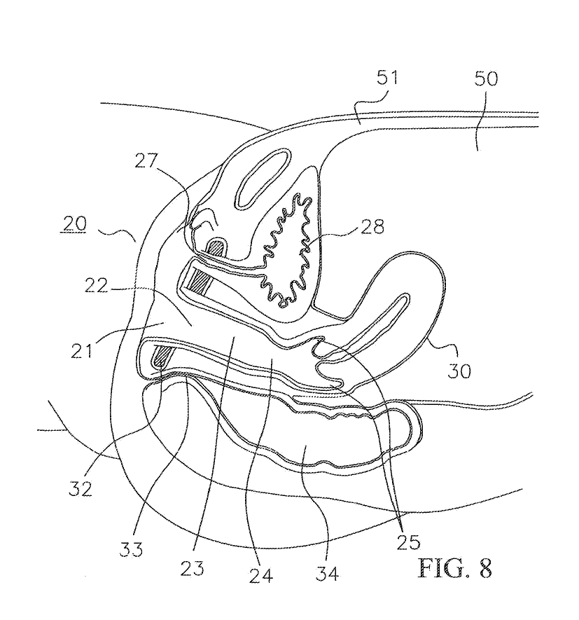

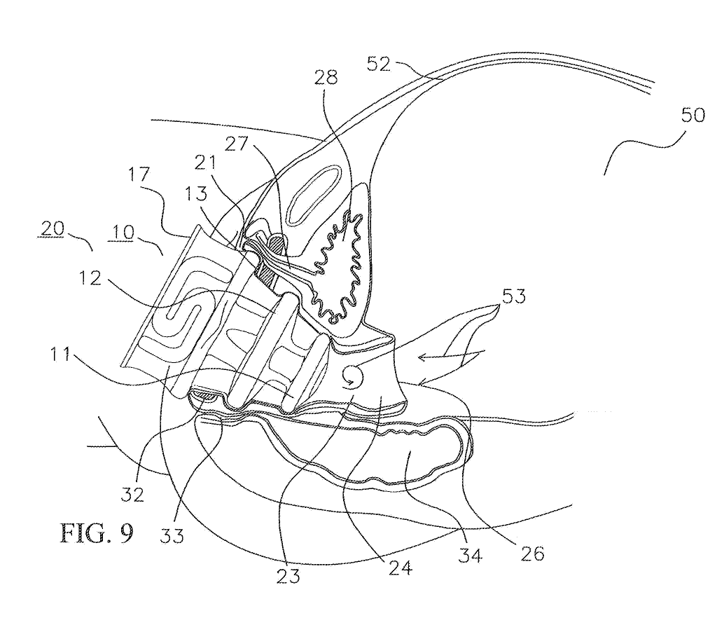

FIG. 1 is a distally oriented perspective view of a hollow compliant anatomic vaginal occluder 10 as presented for use. This device can be made from a variety of surgically acceptable, compliant, biocompatible materials. Examples include plasticized polyvinyl chloride (i.e., Plastisol), silicone rubbers, and thermoplastic elastomers. Manufacturing processes such as plastic injection molding, liquid injection molding, or dip molding can be used to produce the finished device. The anatomic vaginal occluder 10 includes a horizontally oriented distal occlusive prominence 11 preferably an oval shaped prominence having a horizontally oriented major axis which engages an inner-most horizontally oriented zone of a vaginal canal 20. (For anatomic descriptions, see FIGS. 6-9.) An elliptically shaped proximal occlusive prominence 12 preferably an oval shape prominence having a vertically oriented major axis engages a middle, more vertically oriented zone of the vaginal canal 20 just inside of a pelvic floor 32 (shown in FIGS. 8 and 9). A device stop ridge 13 is shaped and sized to preclude an open proximal end 19 of the anatomic vaginal occluder 10 from further entering the external vaginal canal 20 because the stop ridge 13 is too large to pass into a vaginal opening 21. A preferably rounded closed distal end 14 prevents pressurized abdominal gas 53 (FIG. 7) from escaping through the hollow space within the anatomic vaginal occluder 10. The rounded end reduces the likelihood of a suture penetrating the device. A proximal handle 17 for device removal has an open proximal end 19 allows an operator's fingers 41 of an operator's hand 40 (both shown in FIG. 7) to insert and adjust the anatomic vaginal occluder 10 accordingly. A textured gripping surface 18 facilitates gripping at the open proximal end 19 of the occluder 10 to assist in its removal from the vaginal canal 20. Proximal support struts 16 and distal support struts 15 further support the compliant proximal occlusive prominence 12 during its compression within the vaginal canal 20. The distal support struts 15 provide structural support to the distal occlusive prominence 11.

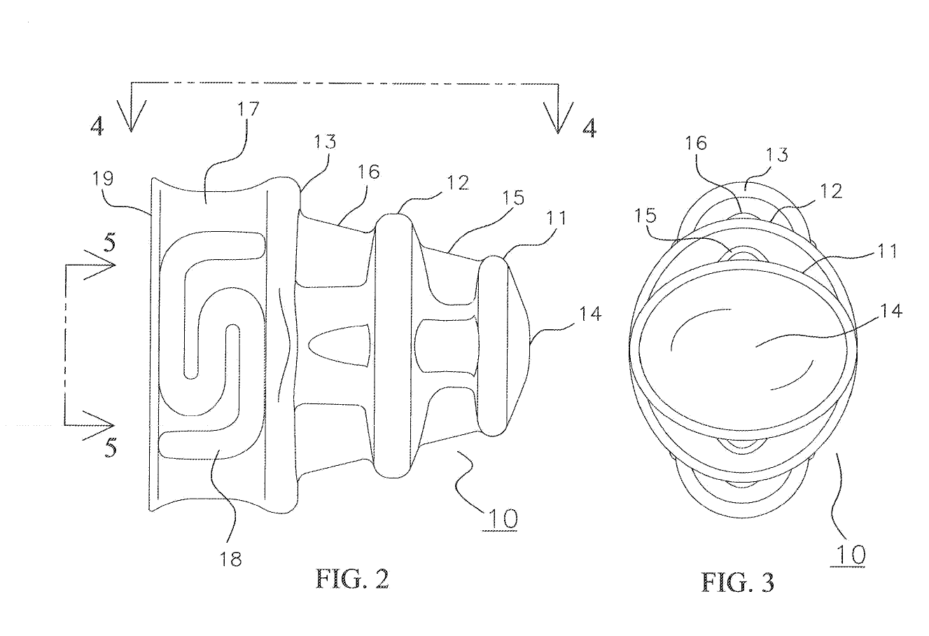

FIG. 2 is a side view of the anatomic vaginal occluder 10 and the associated features presented in FIG. 1. While the distal and proximal occlusive prominences 11 and 12, respectively, are sized so that they can be inserted into the vaginal canal 20, stop ridge 13 is sized to be too large to enter the external vaginal opening 21.

FIG. 3 is a front view of the anatomic vaginal occluder 10 and the associated features presented in FIG. 1. Distal occlusive prominence 11 is seen here as having an elliptical shape, with its long axis oriented side to side or horizontally. The slightly larger proximal occlusive prominence 12 is also elliptical in shape, but its long axis is oriented up and down or vertically. These elliptical orientations correspond to the common natural occurring shape of the human female vaginal canal 20.

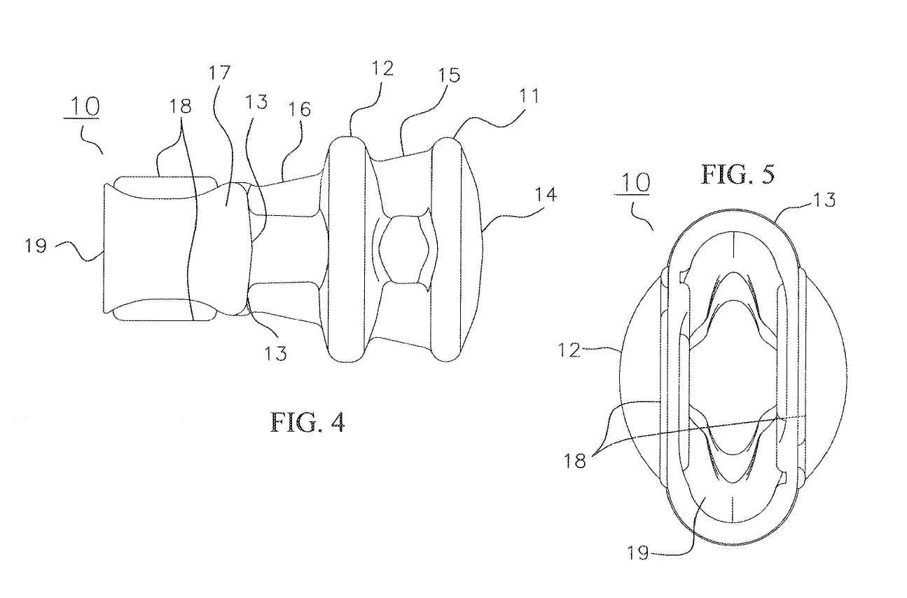

FIG. 4 is a top view of the anatomic vaginal occluder 10 and the associated features presented in FIG. 1, taken along lines 4-4, also of FIG. 2. The proximal and distal support struts 16 and 15 supports the proximal and distal occlusive prominences 12 and 11. The elevated textured gripping surface 18 is seen just distal to the open proximal end 19.

FIG. 5 is a rear view of the anatomic vaginal occluder 10 and the associated features presented in FIG. 1, taken along lines 5-5, also of FIG. 2. The open proximal end 19 is shown along with the textured gripping surface 18. Additionally, the stop ridge 13 along with the proximal occlusive prominence 12 is illustrated.

FIG. 6 is a rear perspective view of the anatomic vaginal occluder 10 and the associated features presented in FIG. 1. Illustrated, from the surgeon's view, is the open proximal end 19, opposite the closed distal end 14, along with the textured gripping surface 18 of the proximal handle 17. Additionally, the stop ridge 13 along with the proximal occlusive prominence 12 and distal occlusive prominence 11, supported by the proximal support struts 16 and distal support struts 15, respectively, are shown.

FIG. 7 is a cross sectional view of the anatomic vaginal occluder 10 and the associated features presented in FIG. 1 with the operator's fingers 41 of the operator's hand 40 shown inserted through the open proximal end 19 ready for application. The fingers 41 provide structural longitudinally and radial support for the hollow, compliant anatomic vaginal occluder 10. Note the fingers 41 act as a supporting mandrel or obturator to enable passage of the device through the confined opening of the pelvic floor 32 (FIGS. 8 and 9), and into the vaginal canal 20.

FIG. 8 is a side view of the human female pelvis sectioned along the midline, also known as a sagittal view, showing an abdomen 50, the vaginal canal 20, the outer opening of vagina 21, the pelvic floor 32 (shown hatched), and a uterus 30 prior to the procedure. An anterior abdominal wall 51 is illustrated without a pneumoperitoneum in its non-pressurized, non-distended state.

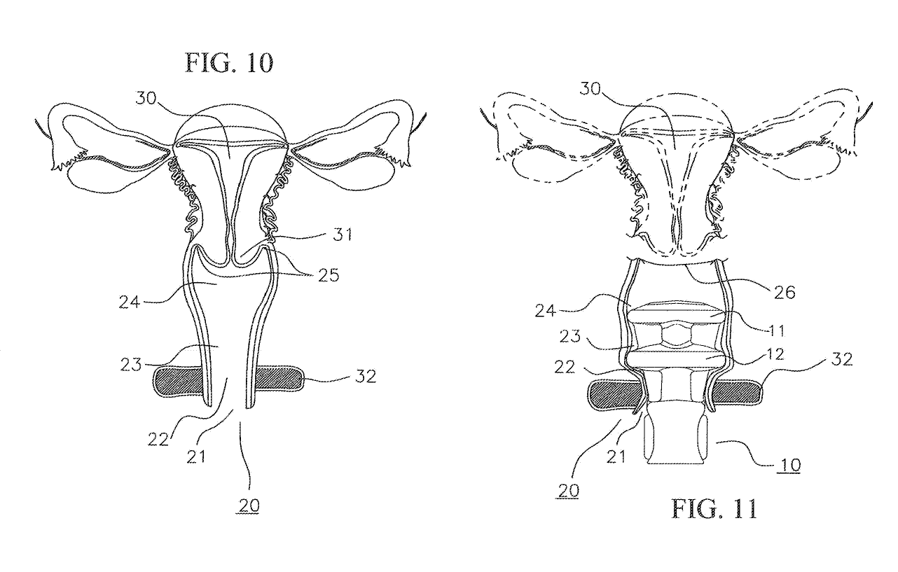

FIG. 9 is a side view sectioned along the midline of the human female pelvis after removal of the uterus 30 (not shown). The anatomic vaginal occluder 10 is shown inserted into vaginal canal 20. The stop ridge 13 of the anatomic vaginal occluder 10 is shown disposed against the skin adjacent to the opening of the vagina 21 with the pelvic floor 32 structure providing a back-stop against further insertion. With the uterus 30 (shown in FIG. 10) removed, insufflation gas 53 from the abdomen 50 that could otherwise pass through an open vaginal cuff 26, is now prevented from leaking by the anatomic vaginal occluder 10. A distended anterior abdominal wall 52 highlights its increased girth due to a pressurized pneumoperitoneum.

FIG. 10 is a sectioned front view, also known as an anterior view, illustrating the vagina 20 with its five described zones starting at the external vaginal opening 21 (zone 1), followed inwardly the vaginal canal 22 (zone 2) then passes through the pelvic floor 32, a vertically oriented middle section of the vaginal canal 23 (zone 3), a horizontal, pre-formiceal section 24 (zone 4) and, finally, a vaginal fornix 25 (zone 5). The uterus 30 prior to the hysterectomy is attached to the vaginal fornix 25 at a uterine cervix 31.

FIG. 11 is a sectioned view similar to HG. 10, which shows the uterus 30 (now ghosted) and the vagina 20 with its external vaginal opening 21 and incised vaginal cuff 26. A top view of the anatomic vaginal occluder 10 is shown with the distal occlusive prominence 11 engaging the inner horizontal section 24 (zone 4) of the vagina 20 and the proximal occlusive prominence 12 engaging the middle vertical section 23 (zone 3) of the vagina 20 near the external vaginal opening 21.

While the invention has been described in connection with several presently preferred embodiments thereof, those skilled in the art will recognize that a number of modifications and changes may be made therein without departing from the true spirit and scope of the invention which accordingly is intended to be defined solely by the appended claims.

1. A vaginal occluder comprising:
a soft resilient body;
an oval shaped distal occlusive prominence on the body having a major axis and a minor axis;
an oval shaped proximal occlusive prominence on the body having a major axis oriented at an angle of approximately 90 degrees to the major axis of the distal occlusive prominence; and
a stop ridge, disposed proximally of the proximal occlusive prominence.

2. The vaginal occluder of claim 1 comprising a cavity extending from the proximal end of the body towards the distal end.

3. The vaginal occluder of claim 1 comprising a textured gripping surface proximal to the stop ridge.

4. The vaginal occluder of claim 2 in which the cavity is sized to receive one or more fingers of an operator.

5. The vaginal occluder of claim 3 comprising a textured gripping surface proximal to the stop ridge.

6. The vaginal occluder of claim 1 comprising a proximal handle having an open proximal end that allows an operator's fingers to insert and adjust the anatomic vaginal occluder

7. The vaginal occluder of claim 1 further comprising proximal support struts and distal support struts for supporting the proximal occlusive prominence during its compression within the vaginal canal.

8. The vaginal occluder of claim 1 in which the prominences are elliptical.

9. The vaginal occluder of claim 1 comprising a closed distal end.

10. A compliant vaginal occluder that is contoured like the human female vaginal canal with a distal occlusive prominence oriented horizontally and a proximal occlusive prominence oriented vertically to enhance vaginal canal occlusion and anatomically orient the vaginal cuff for closure.

11. The compliant vaginal occluder of claim 10 comprising an internal opening to enable insertion into the vaginal canal using the operator's fingers.

12. The compliant vaginal occluder of claim 10 comprising an integrated stop ridge feature that prevents inadvertent complete insertion or too deep insertion into the vaginal canal.

13. The vaginal occluder of claim 1 further comprising a gripping surface to enable simplified removal of the device.

14. The vaginal occluder of claim 10 in which distal tip is rounded.
